(19) United States
(12) Patent Application Publication
(10) Pub. No.: US 2014/0045226 A1
(43) Pub. Date: Feb. 13, 2014

(54) PROCESS FOR TREATING LIGNOCELLULOSIC BIOMASS

(71) Applicant: DAKOTA STAR CAPITAL, LLC, Ames, IA (US)

(72) Inventors: J. Bruce WICKING, Ames, IA (US); Yilin BIAN, Ames, IA (US); Ping LIU, Ames, IA (US); Monlin Kuo, Ames, IA (US)

(21) Appl. No.: 13/937,855

(22) Filed: Jul. 9, 2013

Related U.S. Application Data
(60) Provisional application No. 61/680,997, filed on Aug. 8, 2012.

(51) Int. Cl. D21C 1/06; C12P 7/14; C12P 19/14

(52) U.S. Cl. CPC D21C 1/06; C12P 19/14; C12P 7/14
USPC 435/99; 435/162; 162/14; 426/60

(57) ABSTRACT

A process for treating lignocellulosic biomass is described. The process includes treating particulate biomass with alkali to partially remove hemicellulose sheath on cellulose microfibrils of the biomass; and carrying out an oxidation reaction on the alkali treated biomass to form a treated biomass having a disrupted cellulose crystalline structure. The treated biomass may be further processed to form free sugars and lignin and the free sugars can be used to produce bio-ethanol or used as sugar source in fermentation processes.

### CROSS-REFERENCE TO RELATED APPLICATION

This application claims the benefit of U.S. Provisional Application Ser. No. 61/680,997, filed 8 Aug. 2012, the contents of which are incorporated by reference in their entirety. All patents, patent applications, and publications cited herein are incorporated herein by reference in their entireties.

### BACKGROUND

The technology relates to processes for treating lignocellulosic biomass to allow improved enzymatic hydrolysis of cellulose and hemicelluloses of the biomass. The treated biomass may be further processed to form simple sugars for use in bio-ethanol production. Lignin from the treated biomass may be recovered for further uses.

With an increasing demand for sustainable energy and continuing pressure on greenhouse gas reduction, it is important to develop alternative fuel and chemicals from non-petroleum sources. Lignocellulosic materials, such as wood, agricultural residues and perennial grasses are the most important sources for biofuels production. Based on a report by the Sun Grant Initiative, the United States Department of Energy (USDOE) and United States Department of Agriculture (USDA) have estimated that more than 1.3 billion dry tons of biomass (368 million dry tons of biomass from forestlands and 998 million dry tons from agricultural lands) is available annually in the United States of America. Biomass from agricultural land would produce 428 million dry tons of annual crop residue, 377 million dry tons of perennial crop, and 106 million dry tons of animal manure, processing residues and other miscellaneous sources.

Lignocellulosic materials contain cellulose, hemicelluloses and lignin. Depending on the source chemical composition of biomass is highly variable as shown in Table 1. In general, about 70% of lignocellulosic materials (cellulose and hemicelluloses) can potentially be converted to sugars and fermented to biofuels. Bioconversion of the lignocellulosics involves three major steps: A) Pretreatment of the biomass; B) Conversion of cellulose and hemicelluloses to sugars; C) Conversion of sugars to ethanol via fermentation. Pretreatment of biomass is the key to success of the entire process. The remaining 30% residue is primarily lignin.

TABLE 1
General chemical composition of lignocellulosic materials

| Component | % (dry mass) |
|---|---|
| Cellulose | 30-50 |
| Hemicellulose | 20-40 |
| Lignin | 15-25 |
| Others | 5-50 |

In the case of wood, the plant cell wall is built to provide mechanical support. During cell wall formation, layers of cellulose bundles (microfibrils) sheathed with hemicelluloses are deposited onto the pre-existing wall. At later stages of cell wall formation, lignin precursors infiltrate into compound middle lamella and into micro-voids between microfibrils in secondary cell walls where lignin precursors are polymerized. Consequently, the plant cell wall has a tight structure with a framework of microfibrils embedded in the matrix of hemicelluloses and lignin. Cellulose microfibrils have a cross-section dimension of 300 nm (T)×1,050 nm (W); along the length dimension there are alternating crystalline and amorphous regions. The crystalline region is about 6,000 nm in length where cellulose chains are closely packed so that it is impermeable to water molecules and chemicals. In the amorphous regions, cellulose chains are loosely oriented resulting in greater chemical accessibility. Crystallinity, the volume ratio of microfibrillar crystalline regions, in woody cell walls may be up to 70%. Therefore, without pretreatment cellulose digestibility of plant materials with enzymes is generally less than 30%.

Numerous pretreatment methods have been investigated for bioconversion of lignocellulosic materials. In an extensive review over 180 references were cited and these techniques are summarized in Table 2. However, none of the pretreatment methods has proven to be cost effective on an industrial scale for ethanol production.

TABLE 2
Methods of biomass pretreatment for ethanol production

| | |
|---|---|
| Physical methods | Uncatalyzed steam-explosion |
| | Liquid hot water |
| | Mechanical comminution |
| | High energy radiation |
| Chemical methods | Catalyzed steam-explosion |
| | Acid |
| | Alkaline |
| | Ammonia fiber/freeze explosion |
| | Organosolv |
| | Ionic liquids (Lyocell process) |
| Biological methods | White-rot |
| | Brown-rot |
| | Soft-rot |

A preferred biomass treatment is one that is able to modify the hemicellulose/lignin matrix and to disrupt the crystalline structure of cellulose microfibrils for maximum cellulose saccharification and recovery of native lignin.

As indicated in Table 1 lignin is one of nature's most abundant organic polymers. Lignin makes up to 30% of dry soft wood mass, 20% in hardwood and 20% in grasses. Global production of technical lignin, a by-product in paper pulp production, is around one million tons per year, while majority of pulping spent lignin is burned for energy. Current use of technical lignin is in a variety of low-volume applications such as the use of lignosulfonates in concrete admixtures, dust control during road and mineral ore production and animal feed process aid. The use of kraft lignin is used as filler in plywood adhesives.

Properties of lignin products depend upon their sources and extraction methods. Three monolignols are found in plants: coniferyl alcohol, sinapyl alcohol and ρ-coumaryl alcohol.

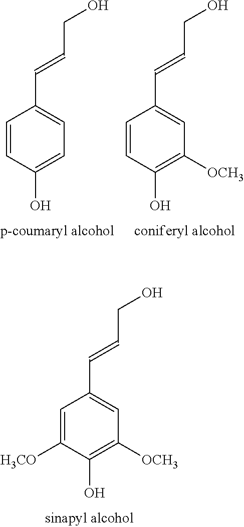 [SMILES: OC/C=C/c1ccc(O)cc1 ; COc1cc(/C=C/CO)ccc1O ; COc1cc(/C=C/CO)cc(OC)c1O]

Lignin in conifers (softwoods) is a polymer of coniferyl alcohol; dicotyledon (hardwoods and other broad leave plants) lignin consists of approximately equal amounts of cnoiferyl and sinapyl alcohols; in addition to cnoiferyl and sinapyl alcohols monocotyledon (grasses, including cornstover and switchgrass) lignin also contains trace ρ-coumaryl alcohol. There are two technical lignins available in the market, lignosulfonates and kraft lignin. Lignosulfonates are obtained from sulfite pulping processes while kraft lignin is obtained from pulping with the sulfate process (kraft pulping). Since the sulfite process is becoming obsolete, availability of lignosulfonates is diminishing. Both lignosulfonates and kraft lignin are forms of highly polymerized lignin; it is difficult to use them directly or to further process them in useful chemicals.

Methods of isolating functional or native lignin from wood have been reported. For mill wood lignin (MWL), it was reported that amount of solubilized lignin could be increased if the finely ground wood meal is treated with hydrolytic enzymes to remove associated polysaccharides prior to solvent extraction. These methods, however, are expensive and time consuming and therefore have not been commercialized.

TABLE 3
Methods of isolating native lignin from wood

| | | Hydrolytic | Organic |
|---|---|---|---|
| Method | Grinding | Enzymes | Solvent(s) |
| Brauns Lignin | No | No | Yes |
| Mill Wood Lignin (MWL) | Yes | No | Yes |
| Cellulolytic Enzyme Lignin (CEL) | Yes | Yes | Yes |

Lignin is an issue in the commercialization of biofuel conversion of lignocellulosic materials. Rather than focusing on removing lignin as required by others, the method disclosed herein focuses on developing a treatment process to allow access to cellulose and hemicelluloses of lingocellulosic biomass.

### SUMMARY

A process is disclosed and described for treating lignocellulosic biomass to allow cellulose and hemicelluloses present in the biomass to be susceptible for hydrolysis to simple sugars and substantially retain native lignin.

Described is a process for treating lignocellulosic biomass which includes:
treating particulate biomass with alkali to partially remove hemicellulose sheath on cellulose microfibrils of the biomass; and
carrying out an oxidation reaction on the alkali treated biomass to form a treated biomass having a disrupted cellulose crystalline structure.

A lignocellulosic biomass suitable for use in the disclosed process can be wood, such as pine and aspen; wood mill waste; bagasse; agricultural residues such as cornstover, cornstalk fiber, wheat and rice straw; perennial grasses such as switchgrass.

Preferably, the particulate lignocellulosic biomass is formed via particle size reduction such as grinding, crushing, milling to a given particle size or range such as 10 to 60 US mesh. Particle size may be any suitable size to allow chemical treatment of the biomass and may be from about 1 to 100 US mesh for example.

Apparatus such as grinders or mills are used to form the particulate lignocellulosic biomass.

A particle size of less than about 100 US mesh is preferred. The particle size can be less than about 60. Particles sizes of about 50, or about 40, or about 30 or about 20 US mesh are suitable. Grinding to a size of about 20 to 40 US mesh has been found to be particularly useful.

One step in the process is generally to remove hemicelluloses partially from the sheath on cellulose microfibrils with a weak alkaline solution such as 1-10% aqueous solutions of sodium hydroxide or ammonium hydroxide.

Preferred alkali treatment includes use of sodium hydroxide at a concentration of about 1 to 10% depending on the raw materials.

Other alkali such as ammonium hydroxide and potassium hydroxide are also suitable.

Concentration of alkaline solutions are adjusted for each different biomass feedstock to maximize hemicellulose removal and minimize lignin extraction.

Other alkali concentrations such as about 0.1 to 1.0% may also be applied.

Alkaline treatment carried out at elevated temperatures has been found to be suitable to partially remove the hemicellulose sheath on the cellulose microfibrils of the biomass. Elevated temperatures of about 50° C. to 80° C. for about 1 to 3 hours have been found to minimize lignin removal. It will be appreciated that lower or higher temperatures such as 20° C., 25° C., 30° C., 35° C., 40° C., 45° C., 50° C., 55° C., 60° C., 65, 70° C., 75° C., 80° C., 85° C., 90° C., 95° C., or 100° C. are within the scope of the disclosed process.

Other treatment times such as 0.5 to 1 hour or 4 and up to 12 hours may be used.

To raise the temperature, heating with steam, hot water, gas heating, oil heating, hot air, electrical heat source or microwave heating are suitable.

After treatment, the alkali may be removed or neutralized by addition of acid. Suitable acids include sulfuric acid, acetic acid and hydrochloric acid.

Subsequently, an oxidation reaction is carried out by a Fenton process using an iron catalyst and hydrogen peroxide.

Preferred Fenton regents are H₂O₂ and FeSO₄. Concentrations of about 0.01 to 2.0 g/g H₂O₂ and about 0.01 to 2.0 M FeSO₄ are suitable. In a preferred embodiment about 0.2 to 0.3 g/g H₂O₂ and about 0.05 to 0.2 M FeSO₄ were used.

The oxidation may be carried out at room temperature or at elevated temperatures.

Elevated temperatures of about 40° C. to 80° C. for about a few to a number of hours have been found suitable. It will be appreciated that lower or higher temperatures such as 20° C., 25° C., 30° C., 35° C., 40° C., 45° C., 50° C., 55° C., 60° C., 65, 70° C., 75° C., 80° C., 85° C., 90° C., 95° C., or 100° C. are within the scope of the disclosed process.

The process may be carried out in suitable reaction vessels such as large mixing tank and enclosed vessels.

The treated biomass has an increased accessibility to action of hydrolytic enzymes to the treated biomass.

After treating with Fenton reagents, treated biomass can be digested with commercial enzymes such as cellulases and hemicellulases. Unlike other methods, the process does not require removal of lignin before enzymatic digestion to produce sugars. Since the two-step treatment is targeted at modification of cellulose crystalline structure, degradation and hydrolysis of lignin is minimal, and thus the subsequent enzymatic digestion of hemicelluloses and cellulose is not affected by the presence of lignin. Other steps may intervene; the disclosure does not limit the process to two steps.

Enzymatic hydrolysis is carried out until the required sugar stream is obtained. Typically this reaction is carried out for a number of days as required.

After 2 to 6 days of enzymatic hydrolysis at conditions recommended by the enzyme manufacturer, up to 93% of the total available cellulose and 20% of hemicellulose may be converted to sugars such as glucose and xylose.

The sugars may be used to produce bio-ethanol by standard fermentation processes.

Examples include production of ethanol from sugar derived from sugar cane and sugar beet and sugar derived from corn starch, milo starch and wheat starch.

In another embodiment, a process of forming sugars from lignocellulosic biomass includes:
treating particulate biomass with alkali to partially remove hemicellulose sheath on cellulose microfibrils of the biomass;
disrupting cellulose crystalline structure of the alkali treated biomass by an oxidation reaction; and
treating the disrupted alkali treated biomass with hydrolytic enzymes to form free sugars and lignin.

The hydrolytic enzymes are preferably selected from cellulases, for example Cellic® CTec series and, hemicellulases Cellic® HTec series from Novozymes.

In one preferred form, the lignin is recovered for further use. The lignin may be recovered by any suitable means such as sedimentation or filtration.

A process of producing alcohol from lignocellulosic biomass includes:
treating particulate biomass with alkali to partially remove hemicellulose sheath on cellulose microfibrils of the biomass;
disrupting cellulose crystalline structure of the alkali treated biomass by an oxidation reaction;
treating the disrupted alkali treated biomass with one or more hydrolytic enzymes to form free sugars and lignin; and
fermenting the free sugars to form alcohol.

One advantage of the described process is that particulate lignocellulosic materials can be treated with dilute alkaline solutions at milder conditions than conventional methods. Treatment with dilute alkaline partially removes hemicellulose sheath on cellulose microfibrils.

An oxidation reaction known as Fenton Reaction, preferably at room temperature, has been found to disrupt the cellulose crystalline structure of alkali treated biomass so as to increase accessibility to hydrolytic enzymes to form sugars.

Enzymes are used to hydrolyze hemicelluloses and cellulose of the treated biomass without the requirement of delignification. The resulting sugar stream and native lignin may be obtained via filtration or by other recovery methods.

The treated biomass is further processed by hydrolytic enzymes to achieve over 90% cellulose saccharification and over 50% native lignin recovery.

The sugar stream, mainly from over 90% saccharification of cellulose, is used to produce bio-ethanol by microbial fermentation. The sugar stream is used in any suitable commercial bio-ethanol production plant.

The cellulolytic enzyme lignin (CEL) or native lignin form the treated biomass may be recovered and is suitable for use as a chemical feedstock. Lignin is used as phenol replacement in phenolic resin, in production of polyurethane foams and in production of vanillin.

It has been found that the sugar stream can also be used to culture a microbial biomass suitable for human of animal feed.

In a further aspect there is provided a process for obtaining a microbial biomass which includes:
treating particulate biomass with alkali to partially remove hemicellulose sheath on cellulose microfibrils of the biomass;
carrying out an oxidation reaction on the alkali treated biomass to form a treated biomass having a disrupted cellulose crystalline structure;
recovering a sugar stream from the treated biomass;
culturing a microbial species on the sugar stream to form a microbial biomass; and
recovering the microbial biomass.

Preferably the microbial biomass is a fungal or yeast biomass.

Preferably the fungal biomass is from an Aspergillus oryzae va., or Aspergillus niger var culture.

Preferably the yeast biomass is from Baker's yeast.

The microbial biomass is suitable for human consumption or as an animal feed or supplement.

Throughout this application, unless the context requires otherwise, the word “comprise”, or variations such as “comprises” or “comprising”, or “includes” will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

In order that the present technology may be more clearly understood, preferred embodiments will be described with reference to the following drawings and examples.

### BRIEF DESCRIPTION OF THE DRAWINGS

FIG. 1 is a schematic of the action of a two-step treatment process on lignocellulosic biomass.

FIG. 2 shows partial hemicellulose extraction of biomass with dilute alkaline.

FIG. 3 shows treatment of fiber with Fenton reagents.

FIG. 4 shows a diagram of treatment process and subsequence enzymatic hydrolysis.

FIG. 5 shows mass balance of 10 grams of hybrid poplar wood subjected to the alkaline-Fenton treatment and enzymatic hydrolysis.

FIG. 6 shows a composition of final solid residue from enzymatic hydrolysis of poplar wood.

FIG. 7 shows mass balance of 10-gram cornstover sample subjected to the alkaline-Fenton treatment and enzymatic hydrolysis.

FIG. 8 shows a process scheme of generating sugar solution from corn stalk and culturing a microorganism to obtain a microbial biomass.

### DETAILED DESCRIPTION

### A. Alkaline-Fenton Pretreatment of Lignocellulosic Materials: a Two-Step Treatment

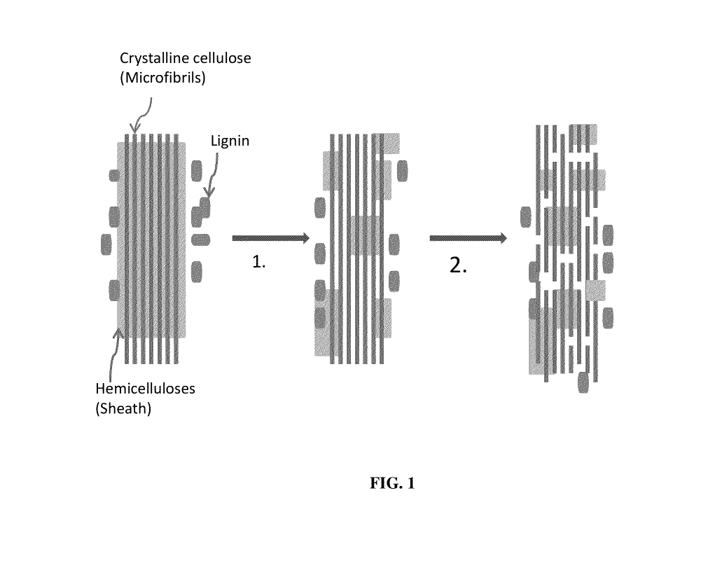

A method for treating particulate lignocellulosic materials with dilute alkaline solutions at milder conditions than used by conventional methods is described. The aim of using dilute alkaline treatment is to partially remove hemicellulose sheath on cellulose microfibrils. A method of treating the alkaline-treated materials with an oxidation reaction known as a Fenton Reaction at room temperature is also described. This oxidation treatment disrupts the cellulose crystalline structure and results in a drastic increase of cellulose accessibility to enzymes. A preferred two-step treatment of lignocellulosic biomass is illustrated in FIG. 1.

### (i) Process for Partial Removal of Hemicellulose to Increase Accessibility to Cellulosic Microfibrils

The first step in the process is to remove hemicelluloses partially from the sheath on cellulose microfibrils with a weak alkaline solution such as 1-10% aqueous solutions of sodium hydroxide and ammonium hydroxide. Unlike other alkaline pretreatments designed to delignify the starting materials, the current process is designed to disrupt the protective hemicellulose sheath on cellulose microfibrils so as to increase cellulose accessibility during further treatment. Concentration of alkaline solutions can be adjusted for each different biomass feedstock to maximize hemicellulose removal and minimize lignin extraction.

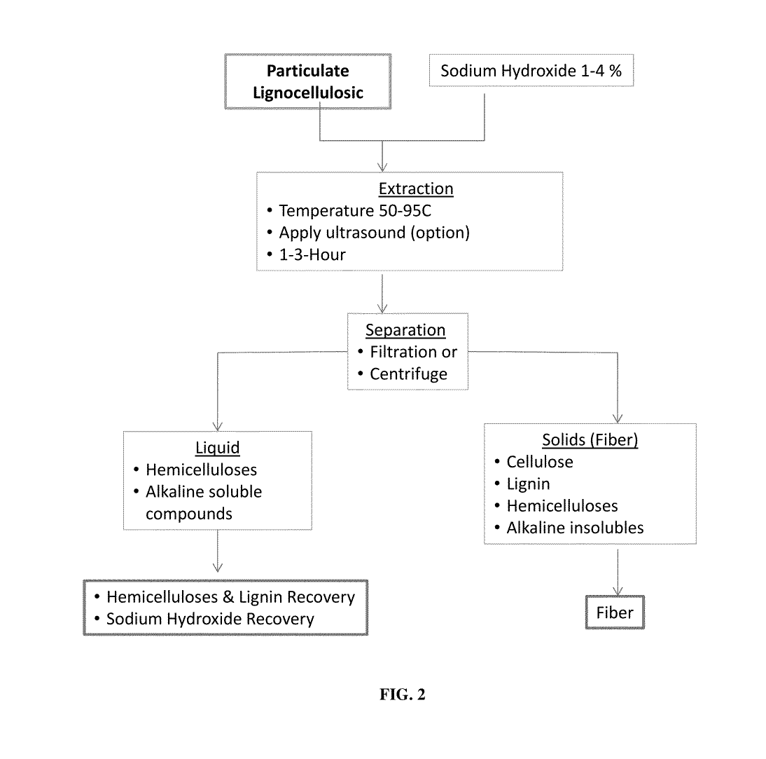

The alkaline treatment is preferably conducted in the range of about 50° C. to 80° C. for about 1 to 3 hours to minimize lignin removal. Alkaline extraction of hemicelluloses at such preferred low temperatures avoids the requirement of pressurized vessels. Low-temperature alkaline hemicellulose extraction also limits hemicellulose hydrolysis so that extracted hemicelluloses can be easily recovered for bio-ethanol production. Use of ultrasound energy can be used to enhance extraction efficiency of sugars and polysaccharides. Therefore, ultrasonic energy may be applied during hemicellulose extraction with dilute alkaline at low temperatures in the current process. The first step of biomass treatment is illustrated in FIG. 2.

### (ii) Process to Decrease Cellulosic Crystallinity for High Efficiency Cellulose Saccharification

Upon exposing cellulose microfibrils by partial removal of hemicelluloses, the material is subjected to oxidative treatment in the second step. Oxidative treatment randomly cleaves glycoside bonds and thereby disrupts the tight cellulose crystalline structure.

Hydrogen peroxide oxidizes ferric ion to generate hydroxyl radical known as Fenton reaction as shown below.

Fe²⁺+H₂O₂—>Fe³⁺+.OH+OH⁻

Fe³⁺+H₂O₂—>Fe²⁺+.OOH+H⁺

Hydroxyl radicals diffuse into cell wall and randomly hydrolyze glycosidic bonds of cellulose in microfibrils.

Preferred regents are H₂O₂ and FeSO₄. Concentrations of 0.01 to 2.0 g/g H₂O₂ and 0.01 to 2.0 M M FeSO₄ can be used. In a preferred embodiment, 0.2 to 0.3 g/g H₂O₂ and 0.05 to 0.2 M FeSO₄ were used.

An improvement is a two-step process in which hemicellulose is removed partially in the first step with alkali treatment followed by oxidative treatment by the use of Fenton reagents, preferably at room temperature. Partial removal of hemicelluloses from the surface of cellulose microfibrils facilitates access of hydroxyl radicals generated from Fenton reagents to disrupt cellulose crystalline structure. Conditions for oxidative reaction with Fenton reagents can be adjusted based on types of biomass being used. The Fenton reaction may be carried out by the use of an electro-Fenton reactor consisting of three electrochemical cells as anodic chamber anode+cathode common chamber and cathodic chamber. The materials would flow through all three cells to be treated. One example of the processing design is demonstrated in Liu 2007. Similar apparatus and equipment of Liu 2007 can be used for the process according to the present invention.

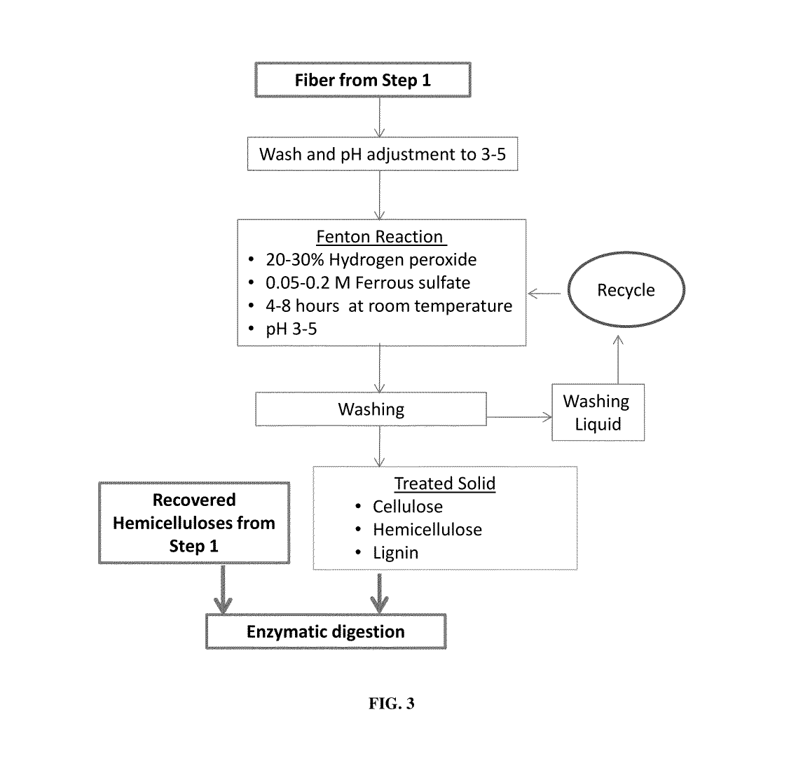

A second process step of the preferred process is illustrated in FIG. 3.

### B. Enzyme Hydrolysis of Treated Fiber to Produce Simple Sugars and Native Lignin

After treating with Fenton reagents biomass fiber is digested with commercial enzymes such as cellulases and hemicellulases. Unlike prior art methods, the process does not require removal of lignin before enzymatic digestion to produce sugars. Since the two-step treatment is targeted at modification of cellulose crystalline structure, degradation and hydrolysis of lignin is minimal, and thus the subsequent enzymatic digestion of hemicelluloses and cellulose is not substantially affected by the presence of lignin.

After 2-6 days of enzymatic hydrolysis at conditions recommended by the enzyme manufacturer, up to 93% of the total available cellulose and 20% of hemicellulose can be converted to glucose and xylose. The solid residue is mainly lignin which has not been subjected to high temperature and strong acids.

### C. Overall Processing of the Pretreatment and Subsequence Enzymatic Hydrolysis

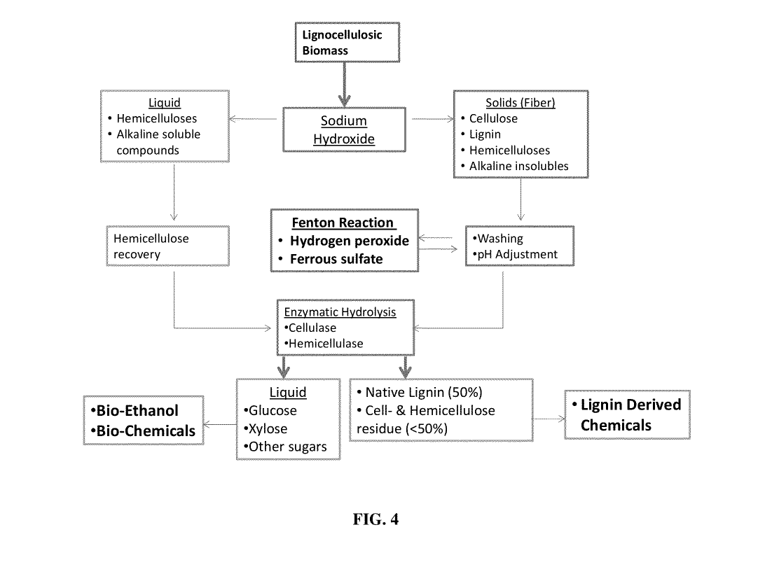

A complete depiction of a preferred embodiment of the process is provided in FIG. 4.

### EXAMPLES

### Example 1

One gram of hybrid poplar wood with bark (dry based) was ground to 20-mesh size. Four treatments were conducted to determine the effect of each process on final yields of glucose and xylose (Table 4). In Treatment 1 wood meal was directly digested with enzymes while wood meal was extracted with 1-2% alkaline at 55-75° C. for 1 h before enzyme digestion. In Treatment 3 wood meal was treated with 0.2-0.3 g/g H₂O₂ and 0.05-0.2 M FeSO₄ followed by enzyme digestion without alkaline extraction treatment, and in Treatment 4 wood meals was treated with alkaline extraction and Fenton reagents before enzyme digestion. Complete hydrolysis of cellulose and hemicelluloses was done with 72% H₂SO₄ to provide references for total available glucose and xylose. Enzymatic digestion was conducted with Dosages of 300 μl cellulase and 6 μl hemicellulase under conditions recommended by manufacturer. HPLC analysis of glucose and xylose were done based on the NREL standard.

TABLE 4
Experiment protocol

| | Acid | | Fenton | | Sugar |
|---|---|---|---|---|---|
| Treatment # | hydrolysis | Alkaline | Reagents | Enzymes | Analysis |
| 1 | — | — | — | Yes | HPLC |
| 2 | — | Yes | — | Yes | HPLC |
| 3 | — | — | — | Yes | HPLC |
| 4 | — | Yes | Yes | Yes | HPLC |
| Control | Yes | — | — | — | HPLC |

Effects of various treatments on percent weight loss and glucose and xylose yield are shown in Table 5. Cellulose and xylan digestibility was calculated as the ratio between glucose and xylose obtained in various treatments and total available glucose and xylose in wood sample. Wood meal directly digested with enzymes without any pretreatment resulted in 11.60% weight loss with 10.02% cellulose and 13.45% xylan digested to glucose and xylose, respectively. When wood meal was extracted with dilute alkaline, 34.04% cellulose and 40.43% xlyan was digested to simple sugars. In Treatment 3, wood meal was treated with Fenton reagents followed by enzymatic hydrolysis, where only 8.61% cellulose and 6.11% xylan was digested. When wood meal was treated with dilute alkaline followed by Fenton reagents cellulose and xylan drastically increased to 79.37% and 71.49%, respectively. The experiment clear demonstrates effectiveness of the low-energy-input and expeditious dilute alkaline/Fenton reaction pretreatment for high efficiency enzyme hydrolysis of cellulose and xlyan. Since efficient cellulose digestibility is the key factor in bio-ethanol conversion, the current invention significantly improves the utilization of wood for biofuels. Other biomass, such as agricultural crop residues and perennial grasses, also can be treated by the process described herein prior to enzymatic digestion.

TABLE 5
Effect of various pretreatment of hybrid poplar wood on enzymatic
digestibility of cellulose and xylan

| | Weight | Glucose | | Xylose | |
|---|---|---|---|---|---|
| | Loss | | Digestibility | | Digestibility |
| | mg/g | mg/g | (%) | mg/g | (%) |
| Treatment 1 | 116.0 | 42.08 | 10.02 | 21.12 | 13.45 |
| Treatment 2 | 435.0 | 142.97 | 34.04 | 63.48 | 40.43 |
| Treatment 3 | 190.5 | 36.15 | 8.61 | 8.85 | 6.11 |
| Treatment 4 | 776 | 329.14 | 78.37 | 112.24 | 71.49 |
| Control | 790 | 420.0 | 157.00 | — | — |

### Example 2

The alkaline extraction-Fenton reaction treatment was further evaluated by using 10-gram hybrid poplar wood samples. The wood samples had the average chemical composition as follows determined by procedures of the National Renewable Energy Laboratory (NREL) (Selig, M. et al. 2008) as set out in Table 6.

TABLE 6
Analysis of polar wood sample

| Component | Amount (%) |
|---|---|
| Hot-water Extract | 8.58 |
| α-Cellulose | 40.48 |
| Hemicelluloses | 29.61 |
| Lignin | 20.97 |
| TOTAL | 100.00 |

Ten grams oven-dried hybrid poplar wood samples (20-mesh particle size) were subjected to the alkaline extraction-Fenton reaction treatment followed by enzymatic digestion in the following conditions:

a) Alkaline Extraction: Extraction of 10-gram samples was conducted in a sonicator with 150 ml 2% aqueous NaOH at 65° C. for 2 hours. The extracts were filtered off by suction and acidified to pH 4.5 to recover hemicelluloses and lignin. The solid residue was washed with filtrates obtained in the subsequent Fenton reaction.
(b) Fenton Reaction: At 1:15 solid to liquid ratio, the solid residue was treated with Fenton reagents containing 20-30% H₂O₂ and 0.05-0.2M Fe₂SO₄ at room temperature and pH 3-5 for 8-12 hours, in which H₂O₂ and Fe₂SO₄ were applied at two different times. The solid residue was filtered off, and the filtrate was used to wash alkaline extracted solid residue.
(c) Enzymatic Hydrolysis: Fenton reagent-treated solids combining hemicelluloses and lignin recovered from alkaline extraction was conducted at 1:10 solid to liquid ratio first with hemicellulase and then with cellulase. Hemicellulase (0.1% to solids) hydrolysis was done at 70° C. and pH 5.0 for 8 hours, and the subsequent cellulase (3.0% to solids) hydrolysis was carried out at 50° C. and pH 5.0 for 3 days

In the step of alkaline extraction, 0.208 g hemicelluloses and 0.319 g lignin were recovered by acidifying alkaline extracts with 4NH₂SO₄ to pH 4.5. The alkaline extracted solid residue was washed with the filtrate obtained after Fenton reaction which helped removing residual NaOH in extracted solids because the filtrate contained substantial amount of acetic acid due to de-acetylation of xylan. Washing alkaline-extracted solids with Fenton reaction filtrate reduces volume of waste water. During enzymatic hydrolysis with hemicellulase, de-acetylation continued and the hydrolysis had to be frequently adjusted to 5.0.

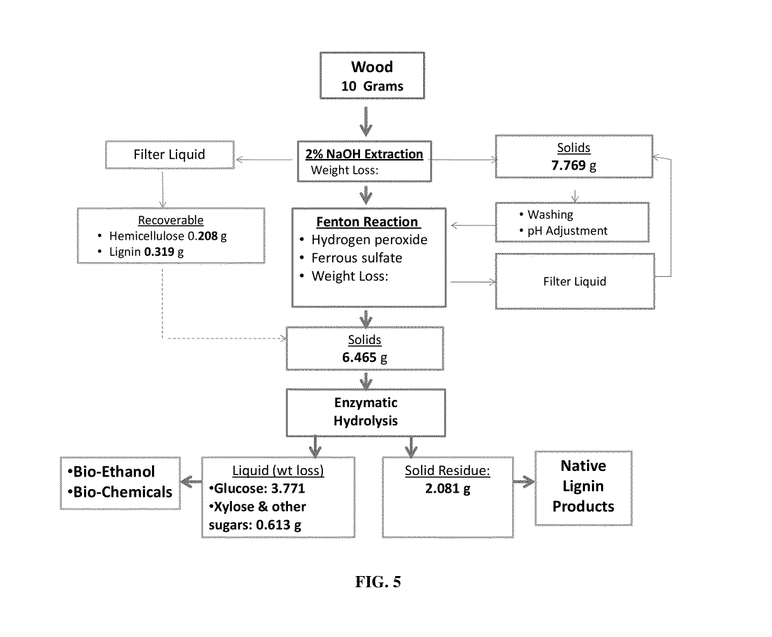

Separated analyses showed that 7.769 g alkaline-extracted solid residue consisted of 1.393 g lignin and 6.375 g holocellulose and that treating alkaline-extracted solid residue with Fenton reagents removed 0.246 g lignin and 1.057 g from holocellulose. Part of the 1.057 g weight loss from holocellulose was release of acetic acid due to de-acetylation of xylan. The subsequent enzymatic hydrolysis of 6.465 g of Fenton reaction solid residue left 2.081 g insoluble residue. These analyses are outlined in FIG. 5.

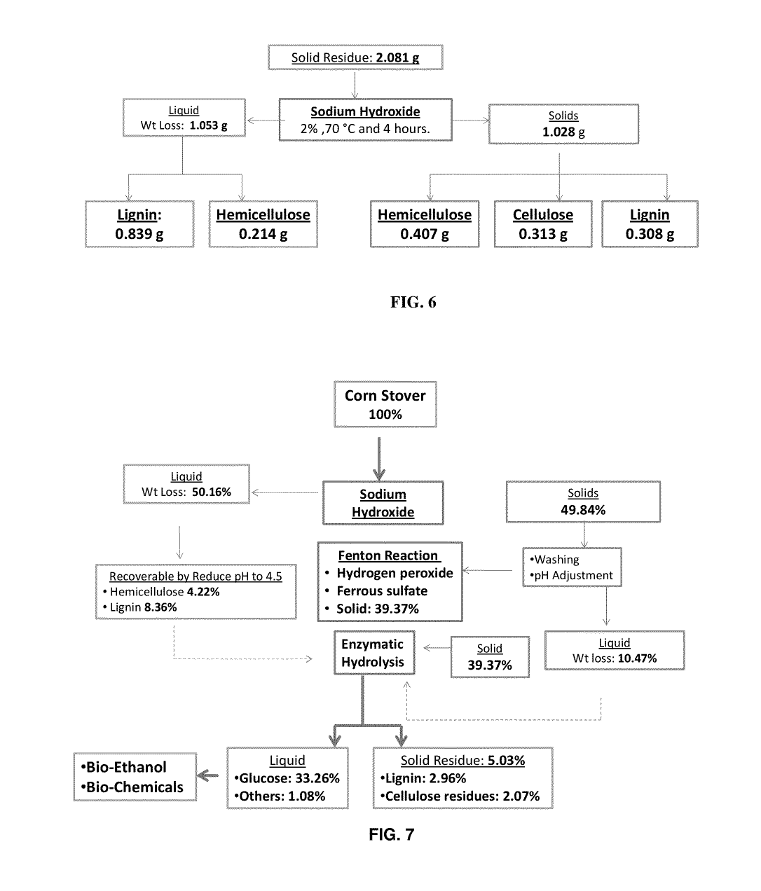

As shown in FIG. 6 extraction of enzymatic hydrolysis solid residue with 2% NaOH at 70° C. for 4 hours obtained 0.839 g lignin and 0.214 g hemicellulose. The final 1.028 g insoluble residue was further analyzed and found to be consisted of 0.313 g cellulose, 0.407 g hemicellulose and 0.308 g lignin. The cell wall lignin is linked to hemicellulose by covalent bonds. Therefore, hemicellulose bound to lignin is not digestible with hemicellulases and the hemicellulose-lignin complex is insoluble to 2% NaOH.

By deduction 3.771 g (4.084 g−0.313 g) of total available cellulose was hydrolyzed to fermentable glucose which translates to 92.34% cellulose digestibility. Only 0.613 g, or 20.70% of the total hemicellulose, was hydrolyzed to fermentable simple sugars. Some hemicelluloses, including water-soluble components, might have hydrolyzed during initial 2% NaOH extraction which could not be recovered by acid precipitation. Also, some hemicellulose were hydrolyzed during treatment with Fenton reagents and lost in the waste water stream. However, 0.208 g and 0.214 g hemicellulose was recovered in the initial and final alkaline extraction, respectively. In the process 1.158 g lignin, or 55.27% of all lignin, was recovered. The recovered lignin is basically native lignin because it has not been subjected to strong acids and high temperatures.

### Example 3

Chemical composition of cornstover in general is similar to that of hardwoods. The average chemical composition of cornstover determined by procedures of the National Renewable Energy Laboratory (NREL) is listed in Table 7 below.

TABLE 7
Analysis of wood cornstover

| Component | Amount (%) |
|---|---|
| Hot-water Extract | 19.66 |
| α-Cellulose | 35.33 |
| Hemicelluloses | 25.86 |
| Lignin | 19.15 |
| TOTAL | 100.00 |

Comparing to poplar wood, cornstover contains less cellulose and water-insoluble hemicelluloses and it also contains much more water-soluble extractives and hemicellulose.

The alkaline extraction-Fenton reaction pretreatment also was evaluated by using 10-gram 20-mesh cornstover samples, and results are outlined in FIG. 7. Ultrasound-assisted extraction with 2% NaOH removed 50.16% dry materials, from which 4.22% hemicellulose and 8.36% lignin were recovered by acidifying the filtrate with 4N H₂SO₄ to pH 4.5. Cornstover is made by volume approximately 25% vesicular bundles where fibers with secondary wall are located and 75% ground tissue (pith). Ground tissue cells possess unlignified thin wall where both lignin in middle lamella and hemicellulose in thin walls are readily accessible to NaOH extraction.

From 49.84% alkaline-extracted residue, 4.20% lignin and 6.27% hemicellulose was removed by Fenton reaction due to degradation of lignin and hydrolysis of hemicellulose which was not recoverable and eventually went to the waste stream. In the subsequent enzymatic hydrolysis of 39.37% solid residue, 34.34% was hydrolyzed to simple sugars leaving 5.03% insoluble residue from which 1.96% lignin and 1.00% hemicellulose was recovered by 2% NaOH extraction. Assuming the 2.07% final solid residue was undigested cellulose, 33.26% of 35.33% available cellulose was hydrolyzed to glucose, a 94.14% cellulose digestibility. From the initial and final 2% NaOH extraction 10.32% native lignin and 5.22% hemicellulose was recovered (FIG. 7).

### Example 4

In this experiment, 10 grams hybrid poplar wood particles (20-mesh) were subjected to the following treatments to produce fermentable sugars for ethanol production.

(a) Alkaline extraction: 2% NaOH at 65° C. for 2 hours; washing and adjusting pH to 5.0.
(b) Fenton reaction: using H₂O₂ at 30 mg and 0.5 ml per gram of wood and 0.05-0.2 M FeSO₄.7H₂O at room temperature for 12 hours; filter, wash and adjust pH to 5.
(c) Enzyme digestion: using hemicellulase (20 μl/g of wood) and cellulase (60 μl) at 50° C. for 5 days; filter out the residue and add 0.5, 1, 1.5 or 2% (NH₄)₂SO₄ to the solution and autoclave at 121° C. for 15 minutes.
(d) Fermentation: Inoculate the final solution with 0.1% dry yeast Saccharomyces cerevisiae at 30° C. for 48 hours.

Results of ethanol production are summarized in Table 8. On the average, glucose concentration in enzymatic hydrolysis filtrates was 7.914 mg/ml. Maximum ethanol yield, 3.602 ml/ml (at 0.791 g/ml=2.849 mg/ml) was obtained when fermentation was conducted with 0.10% dry yeast and 1.0% (NH₄)₂SO₄. Assuming there was 5% glucose consumption for growth of the yeast and at 51.0% theoretic conversion rate, the theoretic ethanol from 7.914 mg/ml glucose would have been 3.033 mg/ml. Therefore, the maximum fermentation efficiency obtained in this study was 93.93%. Such high fermentation efficiency indicates that there is minimal negative effect of the alkaline extraction-Fenton reaction pretreatment on glucose fermentation.

TABLE 8
Bio-ethanol production from alkaline-Fenton-pretreated hybrid poplar
wood

| | | Glucose | Ethanol | Fermentation* |
|---|---|---|---|---|
| Yeast | (NH₄)₂SO₄ | mg/ml | ml/ml | Efficiency (%) |
| 0 | 0 | 7.914 | — | — |
| 0.10% | 0 | 1.669 | 3.213 | 83.97 |
| 0.10% | 0.5 | 1.35 | 3.259 | 84.99 |
| 0.10% | 1 | 1.054 | 3.602 | 93.93 |
| 0.10% | 1.5 | 1.225 | 3.462 | 90.29 |
| 0.10% | 2 | 1.343 | 3.277 | 85.46 |

*Based on 5% glucose consumption for yeast growth and theoretical 0.51 conversion rate of glucose to ethanol (Standard Biomass Analytical Procedures)

### Example 5

From the above experiments, conversion of hybrid poplar wood and cornstover to ethanol and native lignin by the use alkaline extraction-Fenton reaction treatment is summarized in Table 9. Because the treatment results in high cellulose to glucose conversion rate and high fermentation efficiency, 173.09 kg and 152.66 kg ethanol can be produced from glucose alone in wood and cornstover, respectively. These ethanol yields compare favorably to those reported in the literature. Due to the nature of the treatment, substantial amount of hydrolyzed hemicellulose sugars cannot be recovered, but 14.25% and 20.18% of hemicellulose in poplar wood and cornstover, respectively, can be recovered from the 2% NaOH extraction streams. The recovered hemicellulose may be more valuable to be used as feedstock for other chemicals than for ethanol production. Native lignin at greater than 50% yield can be used as feedstock for bio-chemicals.

TABLE 9
Ethanol and native lignin production from hybrid poplar wood and
cornstover

| | Wood | Corn Stover |
|---|---|---|
| Dry Feedstock | 1000 kg | 1000 kg |
| Cellulose content | 40.48% | 35.33% |
| Glucose yield | 377.1 kg | 332.6 kg |
| Ethanol conversion rate | x 51.0% | x 51.0% |
| Fermentation efficiency | x 90.0% | x 90.0% |
| Ethanol yield from glucose | 173.09 kg (218.8 L) | 152.66 kg (193.0 L) |
| Hemicellulose content | 29.61% | 25.86% |
| Fermentable sugars | 61.3 kg | 10.8 kg |
| Ethanol conversion rate | 51.0% | 51.0% |
| Fermentation efficiency | 50.0% | 50.0% |
| Ethanol yield from glucose | 13.6 kg (19.7 L) | 2.75 kg (3.5 L) |
| Recovered from 2% NaOH | 42.2 kg | 52.2 kg |
| stream | | |
| Lignin content | 20.97% | 19.15% |
| Recovered from 2% | 115.8 kg | 100.32 kg |
| NaOH stream | | |
| Native lignin recovery rate | 55.22% | 53.89% |

*Adopted from Standard Biomass Analytical Procedures.

### CONCLUSION

To improve ethanol production, hybrid poplar wood and cornstover were subjected to low-energy input and expeditious pretreatments. Poplar wood and cornstover particles (20-mesh) were first extracted with 2.0% NaOH at 65° C. for 2 h, followed with treating the material with Fenton reagents at room temperature for 12 h. Analysis of enzymatic hydrolysis of pretreated material and subsequent fermentation reached the following conclusions.

By using 2.0% NaOH extraction over 2.0% hemicellulose and 3.0% lignin from poplar wood and 4.0% hemicellulose and 8.0% lignin from cornstover was removed. Partial removal of hemicellulose breaks the protective hemicellulose sheath on cellulose microfibrils.

During Fenton reaction hydroxyl radicals (.OH) were able to infiltrate into cellulose microfibrils to disrupt the cellulose crystalline structure.

Disruption of cellulose crystalline structure allowed over 90% conversion of cellulose to glucose during enzymatic hydrolysis.

Fermentation of enzymatic hydrolysis filtrates indicated over 90% ethanol conversion from glucose.

It is estimated that 173.1 kg (218.8 L) and 152.7 kg (193.0 L) ethanol can be produced from each metric ton of wood and cornstover, respectively.

From each metric ton of wood and cornstover 115.8 kg and 100.3 kg of native lignin can be recovered.

There was low concentration of simple sugars from hemicellulose in enzymatic hydrolysate, but 42.2 kg and 52.2 kg of hemicellulose can be recovered from each metric ton of wood and cornstover, respectively, for other uses.

### Example 6

### A. Production of Corn Fiber Sugar Solution Methods

a. Cornstalk fiber was ground to 20 US mesh.

b. Partial removal of hemicellulose was conducted by either ensilage or weak sodium hydroxide wash. Overall about 25% weight loss had occurred up to this point.

c. Fenton reaction by the use of 0.1-0.5 grams of ferrous sulfate and 0.5-3.0 mL of hydrogen peroxide was applied to a corn fiber-water solution. The addition of hydrogen peroxide could be divided into 2-4 times during 12-24 hours of reaction time.

e. After a 12 to 24 hour treatment by Fenton reagents, the corn fiber was washed, re-dispersed in water and pH adjusted to 5.0 with a 50% acetic acid. In this case, Novozymes Cellic™ CTec cellulase was used, but when using other cellulases, the pH adjustment should match the enzyme manufacturer's specification. The use of hemicellulase could also be applied prior to the cellulase as described in FIG. 7. When other agricultural fiber, such as wheat straw, rice straw and switchgrass are available, they could be treated similarly by the method described prior to enzymatic hydrolysis.

f. A total 1-3% cellulase via fiber weight was applied in two doses during 24-72 hours of incubation in a shake flask at 150 rpm.

g. Corn fiber was visibly reduced in both size and total amount. A 200 US mesh was used to separate the residue fiber from solution.

B. Production of Fungal Biomass from Corn Stalk Sugar Solution Methods

a. The selection of fungi suitable for a such sugar solution includes Aspergillus oryzae var., Aspergillus niger var, or Baker's yeast. All of which are ‘General Regarded as Safe’ (GRAS) so that they can be used as livestock feed or for human consumption.

b. Preparation of an Aspergillus oryzae culture involved the growth of spores on a solid media, generate pre-culture in either synthetic media or the corn stalk sugar solution. The pre-culture was transferred to the production fermentation media to generate a biomass.

c. As the corn stalk sugar solution may not provide complete nutrients for the optimal growth of the fungi, it can be useful to include other nutrients such as a nitrogen source and a phosphors source.

d. Once the final fermentation media was constructed and the pre-culture was introduced, the fermentation was conducted at 30° C., in shake flask at 150 rpm and for 48 hours.

e. The fungal biomass was separated from the fermentor by using two layers of cheesecloth. The dewatered fungal biomass was dried at 50-60° C. over night.

### RESULTS AND CONCLUSIONS

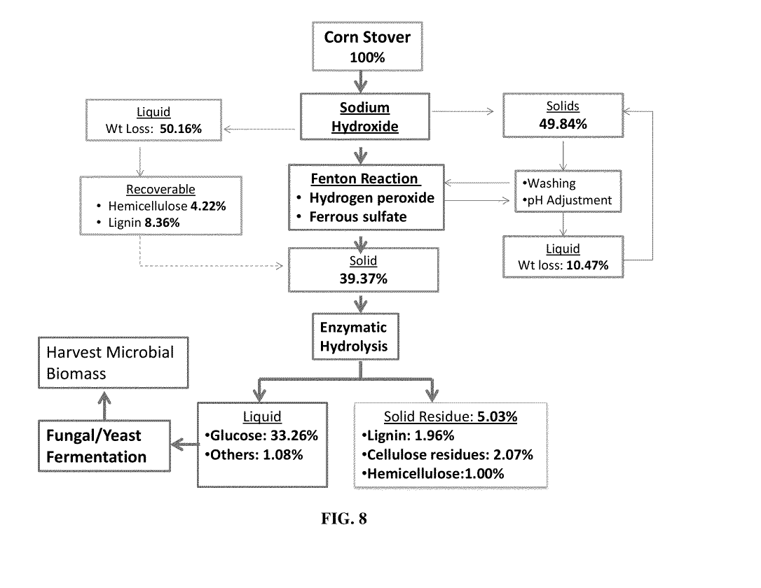

FIG. 8 shows a process scheme of generating sugar solution from corn stalk and culturing a microorganism on the sugar solution to obtain a microbial biomass.

The results of fungal yield in the different corn stalk sugar based media are shown in Table 10.

TABLE 10
Effect of medium composition on solid conversion of corn stalk sugar
solution

| | Dried fungal biomass (g) | Solid |
|---|---|---|
| Media | from 100 g medium | conversion % |
| Corn Stalk Sugar | 0.63 | 31.4 |
| Solution only | | |
| Corn Stalk Sugar | 0.99 | 32.9 |
| Solution with Urea | | |
| Corn Stalk Sugar | 1.04 | 33.1 |
| Solution, Urea, KH₂PO₄ | | |
| and MgSO₄ | | |

The use of Fenton/Enzyme method to generated a corn stalk sugar solution was clearly demonstrated again in this study. The corn stalk sugar solution was used as culture for fungal species to produce edible biomass. The fungi grew well in the corn stalk sugar based media. The use of such a sugar solution as a part of the fermentation media was demonstrated by a total solid conversion over 33% by Aspergillus oryzae.

Converting a non-food source such as cornstalk fiber to a high value feed or food in the form of fungal biomass can assist with addressing world food shortages and may have a large impact in many parts of the world.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### PUBLICATIONS

These publications are incorporated by reference to the extent they relate materials and methods disclosed herein.
Liu, H., et al. 2007. A novel electro-fenton process for water treatment: reaction-controlled pH adjustment and performance assessment. Environ Sci Technol 41: 2937-2942).
Lin, S. Y. and Dence, C. W. (editors) 1992. Methods in Lignin Chemistry, Chapter 3: Isolation and Purification. Springer Series in Wood Science, Springer-Verlag Publisher) as summarized in Table 3.
Selig, M. et al. 2008. Enzymatic Saccharification of lignocellulosic biomass. Technical Report NREL/TP-510-42629.
Zheng Y., et al. 2009. Overview of biomass pretreatment for cellulosic ethanol production. Int. J. Agric & Biol Eng. 2(3): 51-68).

1. A process for treating lignocellulosic biomass, the process comprising:
treating particulate lignocellulosic biomass with alkali to partially remove hemicellulose sheath on cellulose microfibrils of the biomass; and
carrying out an oxidation reaction on the alkali treated biomass to form a treated biomass having a disrupted cellulose crystalline structure.

2. The process according to claim 1 wherein the lignocellulosic biomass is selected from the group consisting of wood, wood mill waste, begass, agricultural residues, cornstover, cornstalk and perennial grasses.

3. The process according to claim 1 wherein the particulate lignocellulosic biomass is formed by grinding, crushing, or milling.

4. The process according to claim 3 wherein the particulate lignocellulosic biomass is from 1 to 100 US mesh.

5. The process according to claim 4 wherein the particulate lignocellulosic biomass is from 20 to 40 US mesh.

6. The process according to claim 1 wherein the alkali is 1 to 10% aqueous solution of sodium hydroxide or ammonium hydroxide.

7. The process according to claim 6 wherein the alkali treatment is carried out at elevated temperatures of about 50° C. to 80° C. for about 1 to 12 hours.

8. The process according to claim 1 wherein the oxidation reaction is carried out by a Fenton process using an iron catalyst and hydrogen peroxide.

9. The process according to claim 8 wherein the iron catalyst is FeSO₄.

10. The process according to claim 9 wherein 0.2 to 0.3 g/g H₂O₂ and 0.05 to 0.2 M FeSO₄ are used at room temperature or at elevated temperatures.

11. The process according to claim 1 further comprising digesting the treated biomass having a disrupted cellulose crystalline structure with one or more enzymes to form free sugars and lignin.

12. The process according to claim 11 wherein the enzyme is selected from the group consisting of cellulase and hemicellulase.

13. The process according to claim 11 further comprising separating the free sugars from the lignin.

14. The process according to claim 13 further comprising carrying out a fermentation reaction on the free sugars to form ethanol.

15. The process according to claim 13 further comprising culturing a microbial species on the free sugars to produce a microbial based ingredient for human consumption or as an animal feed.

16. The process according to claim 15 wherein the microbial based ingredient is fungal or yeast biomass.

17. The process according to claim 16 wherein the fungal biomass is from Aspergillus oryzae va., or Aspergillus niger var.

18. The process according to claim 17 wherein the yeast biomass is from Baker's yeast.
